(19) United States
(12) Patent Application Publication
(10) Pub. No.: US 2022/0285004 A1
(43) Pub. Date: Sep. 8, 2022

(54) System And Method For Connecting Clients With Personal Trainers

(71) Applicant: Jade McArthur, Los Angeles, CA (US)

(72) Inventor: Jade McArthur, Los Angeles, CA (US)

(21) Appl. No.: 16/281,597

(22) Filed: Feb. 21, 2019

(51) Int. Cl. G16H 20/30; G16H 40/67

(52) U.S. Cl. CPC G16H 20/30; G16H 40/67

(57) ABSTRACT

The herein disclosed DeliverFit system and methods for connecting clients with personal trainers can facilitate a client (or user) selecting a personal trainer from a database of pre-screened and certified personal trainers, and can arrange for the selected personal trainer to provide a user-appropriate workout at a location chosen by the user. For example, the user may utilize the herein disclosed methods through a mobile application on the user's phone to arrange for a personal trainer to provide a personalized workout at the user's residence. By utilizing the herein disclosed system and method, persons facing mental, physical, or financial barriers to reaching their fitness goals can be connected with a pre-screened and certified personal trainer that can provide a personalized training session at a time and place that is most convenient. An exemplary embodiment of the methods includes the steps of: receiving enrollment of a plurality of certified personal trainers; receiving enrollment of a client having a residence; receiving an order from the client; selecting an appropriate personal trainer from the plurality of certified personal trainers based upon the order; selecting an appropriate workout based upon the order; arranging for the appropriate personal trainer to provide the appropriate workout at the residence.

### BRIEF DESCRIPTION OF THE INVENTION

The present invention is generally related to fitness and personal training, and more particularly related to systems and methods for connecting a client with one or more personal trainers through a mobile device application. The herein disclosed DeliverFit system and methods can facilitate a user (or client) selecting a personal trainer from a database of pre-screened and certified personal trainers, and can arrange for the selected personal trainer to provide a user-appropriate workout at a location chosen by the user. For example, the user may utilize the herein disclosed methods through a mobile application on the user's phone to arrange for a personal trainer to provide a personalized workout at the user's residence. By utilizing the herein disclosed system and method, persons facing mental, physical, or financial barriers to reaching their fitness goals can be connected with a pre-screened and certified personal trainer that can provide a personalized training session at a time and place that is most convenient.

An exemplary embodiment of the herein disclosed methods includes the steps of: receiving enrollment of a plurality of certified personal trainers; receiving enrollment of a client having a residence; receiving an order from the client; selecting an appropriate personal trainer from the plurality of certified personal trainers based upon the order; selecting an appropriate workout based upon the order; arranging for the appropriate personal trainer to provide the appropriate workout at the residence.

### CROSS-REFERENCES TO RELATED APPLICATIONS

This non-provisional utility application takes priority to the previously filed provisional application: Application No. 62/609,280, filed Dec. 21, 2017, which is hereby incorporated in its entirety by reference.

STATEMENTS AS TO THE RIGHTS TO INVENTIONS MADE UNDER FEDERALLY SPONSORED RESEARCH OR DEVELOPMENT

Not applicable.

REFERENCE TO A “SEQUENCE LISTING,” A TABLE, OR A COMPUTER PROGRAM LISTING APPENDIX SUBMITTED ON A COMPACT DISK.

Not applicable.

### BACKGROUND OF THE INVENTION

According to the Center for Disease Control (CDC), more than 36.5% of US adults have obesity, an unacceptably high number. The estimated annual medical cost of obesity in the US was $147,000,000,000 (147 billion dollars) in 2008 dollars, and the cost per obese individual for medical expenses were nearly $1500 more than for an average weight individual.

There are a multitude of diseases that can be prevented through exercise and nutritional guidance. But many people face obstacles to receiving the proper amount of exercise and to eating properly. People do not know how to exercise and eat properly, or people are unable to travel to see a personal trainer due to a lack of time, mobility, or money.

Personal trainers have extensive knowledge on exercise and nutritional best practices, but it can be difficult for personal trainers to find clients in a world that is saturated with advertisements and subpar wellness advice. There is a need, therefore, for a methodology such as a mobile device application to connect certified and dedicated personal trainers with potential clients desiring improved fitness.

### BRIEF DESCRIPTION OF THE SEVERAL VIEWS OF THE DRAWING

FIG. 1 is a schematic flowchart of a method for connecting a client with one or more personal trainers, in accordance with an exemplary embodiment of the present invention.

### DETAILED DESCRIPTION OF THE INVENTION

The herein disclosed system and methods can facilitate a user (or client) selecting a personal trainer from a database of pre-screened and certified personal trainers, and can arrange for the selected personal trainer to provide a user-appropriate workout at a location chosen by the user. For example, the user may utilize the herein disclosed methods through a mobile application on the user's phone to arrange for a personal trainer to provide a personalized workout at the user's residence. By utilizing the herein disclosed system and method, persons facing mental, physical, or financial barriers to reaching their fitness goals can be connected with a pre-screened and certified personal trainer that can provide a personalized training session at a time and place that is most convenient.

An exemplary embodiment of the methods for connecting a client with one or more personal trainers comprises the steps of: receiving enrollment of a plurality of certified personal trainers; receiving enrollment of a client having a residence; receiving an order from the client; determining one or more appropriate personal trainers from the plurality of certified personal trainers; receiving a personal trainer selection from the client, wherein the personal trainer selection includes one certified personal trainer selected by the client from the one or more appropriate personal trainers; determining one or more appropriate workouts based upon the order; receiving a workout selection from the client, wherein the workout selection includes one workout selected by the client from the one or more appropriate workouts; and arranging for the one certified personal trainer selected by the client to provide the one workout selection by the client at the residence.

As used throughout this specification, the term residence is intended to include a wide variety of venues that may be utilized for the workout provided by the personal trainer. Clients or users may wish to have a personal training session conducted in their home. But clients or users may alternatively wish to have a personal training session conducted at their place of work or at a public venue such as a park or community facility. The term residence, therefore, is intended to include any venue, indoor or outdoor, that may provide the user and the personal trainer with enough physical space to perform the selected workout. In other words, the herein disclosed system and methods can arrange for a personal training session at a venue of the user's choosing—as opposed to the user having to travel to a centralized personal trainer location, such as a gym or training facility.

The terms user and client are used interchangeably throughout the specification. Either term (user or client) is intended to include either one individual user/client or a group of users/clients. The term user or client may refer to the person using the mobile device application, even if this person is using the mobile device application to make arrangement for a training session for a large group; for example, an office manager may utilize the herein disclosed methods with the herein disclosed mobile application system to book one or more training sessions for a group of employees. In this case, the office manager would be the user of the herein disclosed methods, even if the office manager herself is not intending to partake in the training session. As will be apparent, a user may in fact be a group of persons wishing to participate in a group training session.

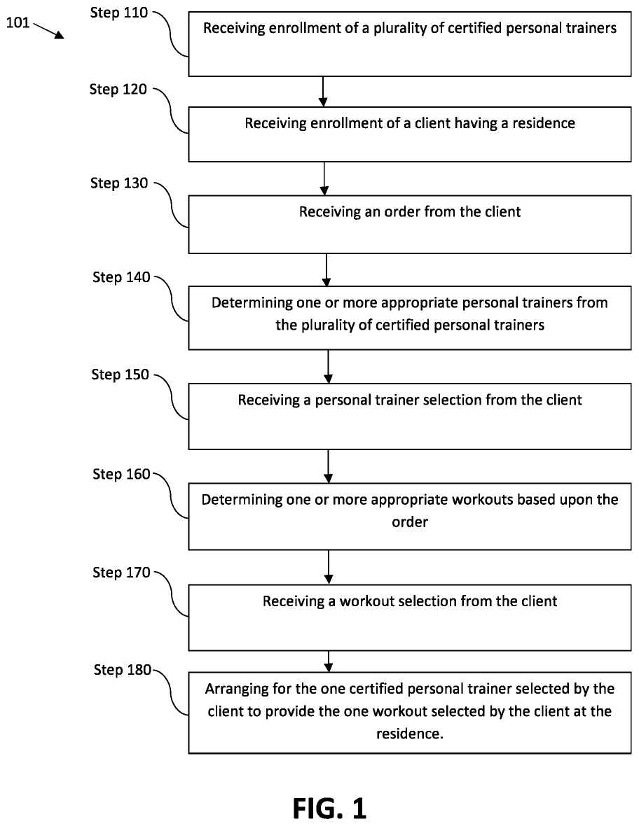

Referring to FIG. 1, method 101 for connection a client with one or more personal trainers includes step 110 receiving enrollment of a plurality of certified personal trainers. Personal trainers are able to sign up with the herein disclosed application in order to become eligible to provide paid personal training sessions to clients. Personal trainers are able to download the application. The application then receives enrollment from one or more personal trainers. In a preferred embodiment, step 110 receiving enrollment of a plurality of certified personal trainers includes verifying that each personal trainer is certified by an established (or verified) personal trainer certification body (such as NASM, ISSA, FASTSTART, etc., for example). In an alternative embodiment, step 110 may further include a certification process (or a professional training process) for aspiring personal trainers to become certified. Once the certification of a personal trainer has been verified (or administered), data representing the personal trainer may be stored in an internet-connected certified personal trainer database that is accessible through the herein disclosed mobile application system.

Method 101 for connected a client with one or more personal trainers further includes step 120 receiving enrollment of a client having a residence. A client wishing to utilize the herein disclosed system and method may start by downloading a mobile application onto a mobile device, such as a smartphone. Step 120 includes receiving enrollment from a client who has already downloaded the application. The application prompts the client to input enrollment information. At a minimum, such enrollment information may include a client identifier (such as a name, for example) and a residence, meaning the venue at which the client wishes to receive a personal training session. For example, step 120 receiving enrollment may include receiving a name and a home address from the client. Those skilled in the art will recognize that additional items of information or data may be received at step 120—such as a client age, a client gender, a client height and weight—and all such additional items of information or data are intended to be included herein at step 120.

Method 101 further includes step 130 receiving an order from the client. The order may be any direction or parameters regarding the type, intensity, or duration of workout that the client wishes to participate in. For example, step 130 may include a type of workout (such as a weightlifting session, or a plyometrics session, for example) and a duration for the workout (such as 60 mins, for example). Step 130 may involve presenting options to the client and receiving selections from the client in the form of a finger tap on the mobile device touchscreen, for example. Alternatively, step 130 may involve presenting questions to the client and receiving answers to the questions through the application.

In an alternative embodiment of the present invention, the order may include a massage (such as a deep tissue massage, for example) or an alternative form of muscular therapy.

Method 101 further includes step 140 determining one or more appropriate personal trainers from the plurality of certified personal trainers. The herein disclosed system and/or mobile device application will utilize client enrollment information in combination with the order to determine a subset of personal trainers (from the overall set of enrolled certified personal trainers) that are appropriate for the client. For example, the geographic location of the residence can be combined with the type, intensity, and/or duration of the training session desired to narrow the overall set of certified personal trainers to a more limited subset of appropriate personal trainers. Those skilled in the art will recognize that various methods of weighing the various items of client enrollment information and various items of order information are possible, and all such methods of weighting the items of information are intended to be included herein. For example, the geographic location of the residence can be weighted most prominently, so that personal trainers located nearby are presented nearer to the top of a list of appropriate personal trainers. Alternatively, the type of training session may be given more weight. For example, an order request for a deep tissue massage may be weighted heavily so that only personal trainers with a background (or certification or training) in massage therapy may be presented as appropriate personal trainers.

Method 101 further includes step 150 receiving a personal trainer selection from the client, wherein the personal trainer selection includes one certified personal trainer selected by the client from the one or more appropriate personal trainers. Step 150 includes receiving an indication of which appropriate personal trainer (as determined in step 140) the client wishes to select. For example, a ranked (or weighted) list of appropriate personal trainers may be presented to the client on the mobile device display screen. In this example, the client would then make a selection of one desired trainer. Step 150 may receive this selection through a finger tap on the display screen of the mobile device as is known in the art, for example.

Method 101 further includes step 160 determining one or more appropriate workouts based upon the order. Step 160 involves utilizing the directions and/or parameters received in the order of step 130 to determine a set of appropriate workouts for the client. For example, if in step 130 the system received order information that the client desires a cross training session, step 160 may determine that the client should be presented with options for a 30-minute cross training session, a 60-minute cross training session, or a 90-minute cross training session. In another alternative example, if in step 130 the system received order information that the client desires massage therapy, step 160 may determine that the client should be presented with options for a 30-minute-deep tissue massage session, a 45-minute hot stone massage session, or a combination strength training and massage session.

After determining one or more appropriate workouts in step 160, method 101 then includes step 170 receiving a workout selection from the client, wherein the workout selection includes one workout selected by the client form the one or more appropriate workouts. Step 170 includes receiving an indication of which appropriate workout (as determined in step 160) the client wishes to select. For example, a ranked (or weighted) list of appropriate workouts may be presented to the client on the mobile device display screen. In this example, the client would then make a selection of one desired workout. Step 170 may receive this selection through a finger tap on the display screen of the mobile device as is known in the art, for example.

Method 101 further includes step 180 arranging for the one certified personal trainer selected by the client to provide the one workout section by the client at the residence. After the herein disclosed system and method has received the client's selection for the certified personal trainer and the client's selection for the workout, arrangements are then made to provide the selected workout by the selected personal trainer at the residence. For example, step 180 may involve sending notification to the one certified personal trainer selected by the client. This notification may include information on the geographic location of the residence, the desired time for the workout, and the type, intensity, and/or duration of the workout. The personal trainer, being an enrolled certified personal trainer, is in agreement to provide the selected workout to the client at the residence. The personal trainer then travels to the residence and provides the workout to the client. As described herein, the residence may be any venue capable of hosting the selected workout and is not intended to be limited to only homes or private residences; for example, the residence may be a public location such as a park or community facility, or may be a place of work such as an office or a school or university.

The present invention may, in some embodiments, include additional features such as nutritional guidance and mentorship programs for children in at-risk communities.

The herein disclosed methods may be implemented through a computer application capable of being downloaded onto a personal computer or a mobile device such as a smartphone or a tablet. In this regard, the present invention may be described as a system for connecting a client with one or more certified personal trainers. Such a system may include an internet-connected database of certified and enrolled personal trainers in communication with a computer application downloadable to a mobile device.

While the present invention has been illustrated and described herein in terms of a preferred embodiment and several alternatives, it is to be understood that the system and methods described herein can have a multitude of additional uses and applications. Accordingly, the invention should not be limited to just the particular description and various drawing figures contained in this specification that merely illustrate a preferred embodiment and application of the principles of the invention.

What is claimed is:

1. A method for connecting a client with one or more personal trainers, comprising the steps of:
receiving enrollment of a plurality of certified personal trainers;
receiving enrollment of a client having a residence;
receiving an order from the client;
determining one or more appropriate personal trainers from the plurality of certified personal trainers;
receiving a personal trainer selection from the client, wherein the personal trainer selection includes one certified personal trainer selected by the client from the one or more appropriate personal trainers;
determining one or more appropriate workouts based upon the order;
receiving a workout selection from the client, wherein the workout selection includes one workout selected by the client from the one or more appropriate workouts;
arranging for the one certified personal trainer selected by the client to provide the one workout selected by the client at the residence.
